(19) United States
(12) Patent Application Publication
(10) Pub. No.: US 2023/0110552 A1
(43) Pub. Date: Apr. 13, 2023

(54) MODEL FOR EVALUATING DEGREE OF LIVER FIBROSIS CONSTRUCTED BASED ON BILE ACIDS

(71) Applicants: Hangzhou Calibra Diagnostics Co., Ltd., Hangzhou (CN); Zhejiang Provincial People's Hospital, Hangzhou (CN)

(72) Inventors: Haijun HUANG, Hangzhou (CN); Xiaofen YUAN, Hangzhou (CN); Rongchang CHEN, Hangzhou (CN); Shanshan CHEN, Hangzhou (CN); Zhouyang KANG, Hangzhou (CN)

(21) Appl. No.: 17/587,940

(22) Filed: Jan. 28, 2022

(30) Foreign Application Priority Data
Oct. 12, 2021 (CN) 2021111888717

(51) Int. Cl. G16C 20/30; G01N 33/74

(52) U.S. Cl. CPC G16C 20/30; G01N 33/743

(57) ABSTRACT

Provided is a model for evaluating the degree of liver fibrosis constructed based on bile acids. A plurality of bile acids are simultaneously detected by a liquid chromatography-tandem mass spectrometer to further improve the accuracy in combination with other liver indicators; moreover, a multiple regression analysis method is applied to establish a grading diagnosis model for the degree of liver fibrosis caused by a chronic liver disease, which can significantly improve the sensitivity and specificity of the existing non-invasive diagnosis of liver fibrosis. When the model is used to evaluate the degree of liver fibrosis of a patient, the highest AUC is up to 0.9278; the sensitivity is up to 86.79%; and the specificity is up to 89.01%. The detection results are completely consistent with the pathological results of clinical liver biopsy. Therefore, patients need not receive a liver biopsy.

### CROSS-REFERENCE TO RELATED APPLICATION

This present application claims the priority of Chinese prior application 202111188871.7, filed on Oct. 12, 2021, the entirety of which is incorporated herein by reference.

### TECHNICAL FIELD

The present invention relates to the field of hepatology, and in particular to a model for evaluating degree of liver fibrosis constructed based on bile acids.

### BACKGROUND

At present, the pHBsAg prevalence rate of general population in China is 5%-6%; there are about 70 million cases of chronic HBV infected people, including about 20-30 million cases of CHB. In 2014, China's Center for Disease Control (CDC) surveyed the prevalence rate among 1 to 29-year-old population throughout the country to find the following result: 1 to 4-year-old (0.32%), 5 to 14-year-old (0.94%) and 15 to 29-year-old (4.38%). HBV-caused liver cirrhosis and HCC (primary hepatocellular carcinoma) in China respectively account for 77% and 84%. For a long time, it is generally believed that liver fibrosis is irreversible, and even a wrong idea of “hepatitis→liver cirrhosis→liver cancer” trilogy has been formed. In recent years, researches have indicated that liver fibrosis is reversible in a particular situation, but if the cause of disease exists persistently, liver fibrosis will develops into irreversible liver cirrhosis. Therefore, the early diagnosis of liver fibrosis and quantification of the degree of liver fibrosis have very important clinical values to the timely intervention treatment and reversal of liver fibrosis development and precaution of liver cirrhosis and liver cancer.

Liver fibrosis is usually a slow and long-term course without obvious symptoms. The “golden criterion” of liver fibrosis examination and staging clinically in China is Liver Biopsy. The method needs to anesthetize a patient and then perform in vivo liver biopsy on the patient with a “biopsy needle” having a length of about 70 mm. This method is invasive and painful in terms of patient experience, and may cause complications and has a higher false negative rate. Patients with liver diseases not only suffer tremendous physical and mental pain in the whole centesis process, but also bear expensive examination fee up to RMB 1500 to RMB 2000 for once, such that lots of patients give up the diagnosis due to the unaffordable examination fee.

In 2019, it was indicated in the Chronic Hepatitis B Prevention Guide that the values of non-invasive diagnosis indicators of liver fibrosis in therapy initiation, efficacy evaluation and predication of long-term outcome are still clinical issues to be studied and solved. The non-invasive diagnosis technologies of liver fibrosis commonly used in clinical practice at present include:

(1) Aspartate transaminase and Platelet Ratio Index (APRI) score: APRI is an indicator which is developed based on the chronic HCV infected data and used for evaluating the degree of HCV-correlated liver fibrosis. Calculating formula: [AST/upper limit of normal, ULN×100/blood platelet count (×10⁹/L); if an adult APRI is ≥2, it prompts the existence of liver cirrhosis; if APRI is <1, liver cirrhosis is excluded. But the recent studies hint that the indicator for evaluating the degree of HBV-correlated liver fibrosis has lower accuracy.

(2) Fibrosis 4 Score (FIB-4): an indicator developed based on the chronic Hepatitis C Virus (HCV) infected data and used for evaluating the degree of HCV liver fibrosis; calculating formula: age (year-old)×AST(IU/L)/[blood platelet count (×10⁹/L)×\sqrt{ALT(IU/L)}; if FIB is ≥3.25, liver fibrosis is diagnosed and liver inflammation grading Matavir score is ≥F3; if FIB is <1.45, Matavir score ≥F is excluded.

(3) Other indicators: extracellular matrix components, such as, hyaluronic acid, procollagen III peptide, collagen IV, and laminin can reflect the occurrence of liver fibrosis, but there is still lack of unified diagnosis critical values for clinical application. γ-glutamyl transpeptidase-P-LCR, red blood cell volume distribution width-P-LCR, red blood cell volume distribution width (%)/blood platelet count (×10⁹/L)] consist of conventional detection indicators; and stable diagnosis critical values are still to be determined. Serum Golgi protein 73 in combination with AST and γ-GT can reflect moderate and severe inflammation of liver. Serum chitinase 3-like protein 1 can predict the moderate and severe liver fibrosis of a patient with normal or slightly increased ALT.

(4) Liver stiffness measurement: liver stiffness measurement (LSM) includes Transient Elastography (TE), Ultrasound-Based Acoustic Radiation Force Impulse (ARFI) and Magnetic Resonance Elastography (MRE). ARFI and MRE technologies are still in the phase of clinical research, and can accurately identify the liver fibrosis in progressive stage and early liver cirrhosis comparatively. But the measured value is influenced by multiple factors, such as, necrotic inflammation of liver, cholestasis and severe fatty liver; and the result interpretation needs to be in combination with patients' bilirubin level and other indicators. There are many interference factors, leading to complex result interpretation.

Liver fibrosis is an inevitable pathological process that various liver diseases develop into liver cirrhosis, and is an only reliable therapy chance to prevent liver diseases from developing into liver cirrhosis. There are two major strategies for the chronic liver diseases around the world: antiviral therapy and prevention of the occurrence of liver fibrosis. The current non-invasive diagnostic techniques for liver fibrosis has low sensibility and specificity in the application of grading diagnosis.

CN112837818A discloses a model for evaluating degree of liver fibrosis of a hepatitis B patient; a regression equation is established by ALT, AST, PTA and LSM to evaluate the degree of liver fibrosis. The model only integrates with the existing diagnostic methods and thus, has low detection sensitivity; AUC is only up to 0.86, and there exists a difference between the true degree of patients' fibrosis and the measured value; the highest sensitivity is 82.5% and the highest specificity is 78.2%.

Therefore, there is lack of a more efficient and accurate non-invasive diagnostic mode of liver fibrosis clinically at present.

### SUMMARY

In view of the problems existing in the prior art, the present invention provides a model for evaluating degree of liver fibrosis constructed based on bile acids. A plurality of bile acids are simultaneously detected by a liquid chromatography-tandem mass spectrometer to further improve the accuracy in combination with other liver indicators; moreover, a multiple regression analysis method is applied to establish a grading diagnosis model for the degree of liver fibrosis caused by a chronic liver disease; the highest AUC is up to 0.928, the detection sensitivity is up to 86.79% and the specificity is up to 89.01%. Therefore, the present invention can significantly improve the sensitivity and specificity of the existing non-invasive diagnosis of liver fibrosis.

The evaluation for the degree of liver fibrosis in this present invention is not to judge whether of liver fibrosis, but judge the severity degree or level of liver fibrosis and judge whether the degree of liver fibrosis is stage 1-2 or 3-4.

The degree grading of a disease is more difficult than the distinguishing of a disease. There exist certain difficulties in the identification of a patient suffering from early/moderate liver fibrosis caused by chronic liver diseases. This is one of the reasons why liver fibrosis cannot be always diagnosed until advanced stage. The grade evaluation of the degree of liver fibrosis is much more difficult than the judgment of liver fibrosis or not.

The evaluation model for the degree of liver fibrosis provided herein can render AUC to be up to 0.928, sensitivity and specificity to be close to 90%, which should be the highest level capable of being achieved in this field at present.

High-specificity and high-sensitivity biomarkers are very important to the early diagnosis and therapeutic effect monitoring of chronic diseases, tumors and other diseases, and are also emphases and difficulties in the development of diagnostic medicine. Bile acid is a group of metabolites of cholesterol in liver decomposition and intestine-liver circulation, and is the final product of cholesterol decomposed and metabolized in liver. Hepatic pathological change always causes the metabolic disorder of bile acids.

The existing documents have reported lots of markers capable of being used for evaluating the presence of liver fibrosis. But in these markers, which kind of marker can be really used for the grade evaluation of liver fibrosis is still the difficulty of the current researches.

By a large number of studies, this research group finds that the change of bile acid concentration in a serum specimen is closely associated with the degree of liver fibrosis, and picks out the bile acid with a higher degree of influence to construct a grading diagnosis model for the degree of liver fibrosis.

The occurrence and development courses of chronic diseases, tumors and other diseases are always accompanied by the abnormal changes in biochemical metabolic pathways of a human body. Liquid Chromatography-Tandem Mass Spectrometry (LC-MS/MS) is a core technology to the research on targeting metabolic pathways and has wide application prospects and basis in clinical examination. To develop a standardized kit suitable for the LC-MS/MS examination method is beneficial to medical detection institutions to promote the detection accuracy and standardization of small-molecular biomarkers.

The present invention establishes metabolic phenotype database of patients with chronic liver diseases at different degrees of liver fibrosis based on a technology of bile acid detection by LC-MS/MS, and uses a multiple regression analysis method to establish a data model for the grading diagnosis of liver fibrosis and an aided decision making system, which provides a new approach for the detection of liver fibrosis and liver cirrhosis. Further, the present invention can help a large number of patients with chronic liver diseases achieve early diagnosis, early prevention and standardized treatment, thereby blocking the course of disease of “chronic liver disease/hepatitis-liver fibrosis-liver cirrhosis-liver cancer”, benefiting more people.

In one aspect, the present invention provides a method for evaluating degree of liver fibrosis, and the method evaluates a degree of liver fibrosis by detecting a concentration of a bile acid.

Liver histopathologic diagnosis is reference to the consensus on the evaluation of diagnosis and therapeutic effect of liver fibrosis in 2002; and the liver histopathologic fibrosis includes five stages, namely, S0, S1, S2, S3 and S4.

The evaluation on the degree of liver fibrosis in this present invention refers that the people with stage 1-2 liver fibrosis (S0-S2) and the people with stage 3-4 liver fibrosis (S3-S4) are exactly distinguished from hepatic tissue population based on the markers showing different degrees of liver fibrosis.

The bile acid in this present invention includes: glycocholic acid (GCA), taurocholic acid (TCA), chenodesoxycholic acid (CDCA), glycochenodeoxycholic acid (GCDCA), taurochenodeoxycholic acid (TCDCA), deoxycholic acid (DCA), glycodesoxycholic acid (GDCA), taurodeoxycholic acid (TDCA), lithocholic acid (LCA), glycocholic acid (GLCA), taurolithocholic acid (TLCA), ursodeoxycholic acid (UDCA), glycoursodeoxycholic acid (GUDCA), tauroursodeoxycholic acid (TUDCA), cholic acid, and the like.

Through a large number of clinical studies, the inventor proves that the concentration of bile acids is highly correlated to the degree of liver fibrosis; and the correlation is obviously greater than that between other substances and the degree of liver fibrosis. That is, the people with stage 3-4 liver fibrosis (S3-S4) has more distinct change rule in the concentration of bile acids relative to the people with stage 1-2 liver fibrosis (S0-S2). Therefore, the people with stage 1-2 liver fibrosis (S0-S2) and the people with stage 3-4 liver fibrosis (S3-S4) can be exactly distinguished by detecting the concentration of bile acids.

The research and analysis prove that in general, the correlation between the concentration of bile acid and the degree of liver fibrosis is generally higher than that of other substances; moreover, different bile acids have different correlations to the degree of liver fibrosis; and certain bile acids have particularly high correlations to the degree of liver fibrosis.

The correlation between the concentration of bile acid and the degree of liver fibrosis can be represented by an OR value in statistical analysis. Certainly, the correlation may be distinguished by a βvalue, a p-value, or the like; OR value is the most visualized and distinct. For example, LCA has an OR value being up to 5.643×10¹⁶ which is 10¹⁶ folds of other bile acids; with such a high correlation, it can be understood that LCA concentration is detected independently to judge the degree of liver fibrosis; for another example, TUDCA has an OR value also being up to 1.864×10⁷ which is 10⁷ folds of other bile acids; GLCA has an OR value being up to 1.0958×10⁶, followed by UDCA, TCDCA, GCA, and TCA. The single detection of the concentration of a bile acid with a high correlation can be completely used to judge the degree of liver fibrosis, thus distinguishing the people with stage 1-2 liver fibrosis (S0-S2) and the people with stage 3-4 liver fibrosis (S3-S4).

Definitely, any bile acid with a high OR value may be picked to jointly establish a model for judging the degree of liver fibrosis, and the accuracy may be higher. Even some other markers may be also added to further improve the accuracy. It is simple and practicable to use one or more bile acids to judge the degree of liver fibrosis; and the simple course may be also used for the preliminary and temporary evaluation.

Further, the bile acid is selected from any one or more of GCA, TCA, CDCA, GCDCA, TCDCA, DCA, GDCA, TDCA, LCA, GLCA, TLCA, UDCA, GUDCA, TUDCA and CA.

Researches prove that the correlation between the concentration of bile acid and the degree of liver fibrosis is generally higher than that of other substances. Therefore, the single detection of the concentration of a bile acid with a high correlation may be used for distinguishing the degree of liver fibrosis.

Further, the bile acid is selected from any one or more of UDCA, TUDCA, LCA, GLCA, GCA and TCDCA.

In bile acids, the correlation between the concentration of any of UDCA, TUDCA, LCA, GLCA, GCA and TCDCA and the degree of liver fibrosis is higher than that of other bile acids. Therefore, the degree of liver fibrosis can be distinguished by independently detecting the concentration of any one of UDCA, TUDCA, LCA, GLCA, GCA and TCDCA.

Further, the bile acid is selected from any one or more of LCA, TUDCA and GLCA.

LCA, TUDCA and GLCA have particularly high correlations to the degree of liver fibrosis, of which, LCA has the highest correlation with an OR value being up to 5.643×10¹⁶ and a very high weight; the second one is TUDCA with an OR value being up to 1.864×10⁷, then followed by GLCA with an OR value being up to 1.0958×10⁶. It can be understood that the degree of liver fibrosis can be judged by independently detecting the concentration of any one of LCA, TUDCA and GLCA.

Further, the bile acid is selected from any one or more of LCA and TUDCA.

LCA and TUDCA respectively rank first and second in the correlation to the degree of liver fibrosis and have a very high weight. Therefore, LCA and TUDCA can be independently detected to distinguish the degree of liver fibrosis.

Further, the bile acid is selected from LCA.

In numerous bile acids, LCA has the highest correlation to the degree of liver fibrosis; that is, it is definitely most accurate to distinguish the degree of liver fibrosis by independently detecting the value of LCA.

Further, a regression equation is constructed to calculate a predicted value of liver fibrosis, thus evaluating the degree of liver fibrosis, and the regression equation is as follows: BAS=43.18×LCA+16.74×TUDCA+13.91×GLCA+5.57×UDCA+0.89×TCDCA+0.77×GCA+0.21×TCA+0.1×CDCA+0×TDCA−0.08×GDCA−0.16×GCDCA−0.43×CA−3.29×GUDCA−4.39×DCA−35.19×TLCA, in which, BAS represents a predicted value of liver fibrosis.

Further, a regression equation is constructed to calculate a predicted value of liver fibrosis, thus evaluating the degree of liver fibrosis, and the regression equation is as follows: BASA=61.92×LCA+38.53×TUDCA+4.66×UDCA+2.14×TCA+0.9×GDCA+0.42×DBIL+0.27×RBC+0.26×GCDCA+0.24×CDCA+0.22×GLU+0.19×WBC+0.03×GGT+0.02×ALT+0.02×TBIL+0.01×AST−0.04×IBIL−0.05×TDCA−0.16×CA−0.67×GCA−0.8×GLCA−1.51×TCDCA−3.77×GUDCA−5.96×DCA−16.07×TLCA, in which, BASA represents a predicted value of liver fibrosis.

Further, the regression equation has a value of BAS or BASA; when BAS is greater than 0.63, the liver fibrosis is a stage 3-4 liver fibrosis; when BAS is lower than 0.63, the liver fibrosis is a stage 1-2 liver fibrosis; when BASA is greater than 0.63, the liver fibrosis is a stage 3-4 liver fibrosis; and when BASA is lower than 0.63, the liver fibrosis is a stage 1-2 liver fibrosis.

In a further aspect, provided is a system for evaluating degree of liver fibrosis; the system includes a data input/output interface and a data analysis unit, where the data input/output interface inputs a concentration of at least one bile acid, after the concentration is analyzed by the data analysis unit, the data input/output interface outputs an evaluation result of the degree of liver fibrosis.

Further, the bile acid includes any one or more of GCA, TCA, CDCA, GCDCA, TCDCA, DCA, GDCA, TDCA, LCA, GLCA, TLCA, UDCA, GUDCA, TUDCA and CA.

Further, the bile acid includes any one or more of UDCA, TUDCA, LCA, GLCA, GCA and TCDCA.

Further, the bile acid includes any one or more of LCA, TUDCA and GLCA.

Further, the bile acid includes any one or more of LCA and TUDCA.

Further, the bile acid includes LCA.

Further, the data analysis unit calculates a predicted value of liver fibrosis based on the regression equation, thus evaluating a degree of liver fibrosis.

Further, the regression equation is as follows: BAS=43.18×LCA+16.74×TUDCA+13.91×GLCA+5.57×UDCA+0.89×TCDCA+0.77×GCA+0.21×TCA+0.1×CDCA+0×TDCA−0.08×GDCA−0.16×GCDCA−0.43×CA−3.29×GUDCA−4.39×DCA−35.19×TLCA, in which, BAS represents a predicted value of liver fibrosis.

In the regression equation constructed herein, as LCA has the highest correlation to the degree of liver fibrosis, the weight is inevitably up to the maximum, accordingly, LCA has the highest coefficient (43.18) in the equation, followed by TUDCA and GLCA. As can be seen, the concentration coefficients of various bile acids in the regression equation also mean the level of correlation between the bile acid and the degree of liver fibrosis.

Further, the data input/output interface further needs to input a concentration/concentrations of one or more of DBIL, RBC, GLU, WBC, GGT, ALT, TBIL, AST and IBIL.

To further improve the detection sensitivity and specificity, on the basis of bile acids, the present invention further adds the concentrations of direct bilirubin (DBIL), red blood cell count (RBC), fasting blood-glucose (GLU), white blood cell count (WBC), glutamyl transpeptidase (GGT), alanine transaminase (ALT), total bilirubin (TBIL), aspartate transarninase (AST), and indirect bilirubin (IBIL). After through a large number of studies, a grading diagnosis model for the degree of liver fibrosis with AUC of 0.928, detection sensitivity of 86.79% and specificity of 89.01% is established finally.

Further, the regression equation is as follows: BASA=61.92×LCA+38.53×TUDCA+4.66×UDCA+2.14×TCA+0.9×GDCA+0.42×DBIL+0.27×RBC+0.26×GCDCA+0.24×CDCA+0.22×GLU+0.19×WBC+0.03×GGT+0.02×ALT+0.02×TBIL+0.01×AST−0.04×IBIL−0.05×TDCA−0.16×CA−0.67×GCA−0.8×GLCA−1.51×TCDCA−3.77×GUDCA−5.96×DCA−16.07×TLCA, in which, BASA represents a predicted value of liver fibrosis.

Further, the regression equation has a value of BAS or BASA; when BAS is greater than 0.63, the liver fibrosis is a stage 3-4 liver fibrosis; when BAS is lower than 0.63, the liver fibrosis is a stage 1-2 liver fibrosis; when BASA is greater than 0.63, the liver fibrosis is a stage 3-4 liver fibrosis; and when BASA is lower than 0.63, the liver fibrosis is a stage 1-2 liver fibrosis.

In a further aspect, provided is use of the system or equation described above in evaluating the degree of liver fibrosis.

In a further aspect, provided is use of bile acids in the construction of a model for evaluating degree of liver fibrosis.

Further, the bile acid is selected from any one or more of GCA, TCA, CDCA, GCDCA, TCDCA, DCA, GDCA, TDCA, LCA, GLCA, TLCA, UDCA, GUDCA, TUDCA and CA.

Further, the concentration of the bile acid is measured by a liquid chromatography-tandem mass spectrometer.

A patent “method for detecting 17 bile acids in blood serum by high-performance liquid chromatography-tandem mass spectrometry” (application No.: CN2020101870483) has been applied for the method for simultaneously detecting a plurality of bile acids by a liquid chromatography-tandem mass spectrometer used in this present invention. The detection technology and a multiple regression analysis method are used to successfully establish a grading diagnosis model for the degree of liver fibrosis caused by a chronic liver disease with higher sensitivity and specificity. In this model, the AUC can be up to 0.834, the detection sensitivity can be up to 75.47% and the specificity can be up to 76.92%.

In a further aspect, provided is a method for establishing the model described above, including the following steps:

(1) obtaining a liver tissue specimen and performing pathological fibrosis staging of the liver tissue, then dividing into a stage 1-2 group of liver fibrosis and a stage 3-4 group of liver fibrosis according to the staging results;

(2) collecting the concentrations of bile acids, DBIL, RBC, GLU, WBC, GGT, ALT, TBIL, AST and IBIL from the two groups of patients; where, the bile acid is selected from one or more of GCA, TCA, CDCA, GCDCA, TCDCA. DCA. GDCA. TDCA, LCA, GLCA, TLCA, UDCA, GUDCA, TUDCA and CA;

(3) examining the correlation of DBIL, RBC, GLU, WBC, GGT, ALT, TBIL, AST, IBIL and bile acids to the significant occurrence of liver fibrosis;

(4) using a multiple analysis method to judge the patient's degree of liver fibrosis, and performing regression analysis on the all detection results to establish a same grading mathematical model for liver fibrosis, and using a calibration curve and ROC curve method to evaluate the efficiency of the regression model.

Further, the present invention analyzes the values of albumin (ALB), alkaline phosphatase (ALP), hemoglobin (HGB), liver stiffness measurement (LSM), C-reaction protein (CRP), globulin (GLB), ratio of albumin of liver function to globulin=albumin/globulin (A/G), alpha fetoprotein (AFP), blood platelet (PLT), prothrombin time (PT), prothrombin activity (PTA), and total bile acid (TBA), as well as other multiple bile acids, such as, HCA and HDCA, and calculates the values of APRI and FIB.4.

Further, the concentration of the bile acid is detected by liquid chromatography-tandem mass spectrometry; the ALB, ALT, AST, GGT, ALP, TBIL, DBIL, IBIL, GLU, RBC, WBC and HGB are detected by a biochemical analyzer; PLT is detected by a cell analysis meter; then the values of APRI and FIB.4 are calculated.

Further, the regression analysis is a LASSO regression analysis method.

Further, the correlation examination in the step 3) has the following specific steps: LASSO regression analysis is performed on DBIL, RBC, GLU, WBC, GGT, ALT, TBIL, AST, IBIL, ALB, ALP, HGB, LSM, CAP, A/G, AFP, PLT, PT, PTA, TBA, and bile acids to find out the variables with high weight.

Further, inclusion and exclusion criteria of the independent variables in step 4) are respectively P<0.05 and P>0.10; the regression model efficiency is evaluated by a calibration curve and a ROC curve method.

In a further aspect, provided is a method for evaluating degree of liver fibrosis, and the degree of liver fibrosis is evaluated mainly by detecting concentrations of a plurality of bile acids in a serum specimen and by the above model respectively as well as calculating the value of the regression equation. The bile acid is selected from one or more of GCA, TCA, CDCA, GCDCA, TCDCA, DCA, GDCA, TDCA, LCA, GLCA, TLCA, UDCA, GUDCA, TUDCA and CA.

Further, the concentrations of bile acids in the serum specimen are detected by liquid chromatography-tandem mass spectrometry; when the model of the BASA in claims is taken, the concentrations of DBIL, RBC, GLU, WBC, GGT, ALT, TBIL, AST and IBIL further need to be analyzed.

Further, the concentrations of DBIL, RBC, GLU, WBC, GGT, ALT, TBIL, AST and IBIL are detected by a biochemical analyzer.

Further, the regression equation has a value of BAS or BASA; when BAS is greater than 0.63, the liver fibrosis is a stage 3-4 liver fibrosis; when BAS is lower than 0.63, the liver fibrosis is a stage 1-2 liver fibrosis; when BASA is greater than 0.63, the liver fibrosis is a stage 3-4 liver fibrosis; and when BASA is lower than 0.63, the liver fibrosis is a stage 1-2 liver fibrosis.

In a further aspect, provided is use of bile acids in the construction of a model for evaluating degree of liver fibrosis.

Further, the bile acid is selected from any one or more of GCA, TCA, CDCA, GCDCA, TCDCA, DCA, GDCA, TDCA, LCA, GLCA, TLCA, UDCA, GUDCA, TUDCA and CA.

In a further aspect, provided is use of a detection kit for evaluating degree of liver fibrosis; the detection kit includes a detection reagent for detecting bile acids by liquid chromatography-tandem mass spectrometry.

Further, the bile acid is selected from any one or more of GCA, TCA, CDCA, GCDCA, TCDCA, DCA, GDCA, TDCA, LCA, GLCA, TLCA, UDCA, GUDCA, TUDCA and CA.

Further, the detection kit includes a mobile phase A (0.01% formic acid-5 mM ammonium acetate aqueous solution), a mobile phase B (methanol solution), bile acid standard, isotope internal standards of bile acid, and the like.

The present invention establishes a model for evaluating liver fibrosis and has the following beneficial effects:

(1) the content of a plurality of bile acids are simultaneously detected by liquid chromatography-tandem mass spectrometry; and a multiple regression analysis method is taken to establish a grading diagnosis data model for liver fibrosis caused by chronic liver diseases, and to develop an aided decision making system for non-invasive diagnosis of liver fibrosis;

(2) the present invention is further in combination with indicators DBIL, RBC, GLU, WBC, GGT, ALT, TBIL, AST and IBIL to construct an effective and accurate model for evaluating degree of liver fibrosis; and the AUC is up to 0.9278; the sensitivity is up to 86.79% and the specificity is up to 89.01%;

(3) the present invention is convenient and fast; the detection results are completely consistent with the pathological results of clinical liver biopsy. Therefore, patients need not receive a liver biopsy.

### BRIEF DESCRIPTION OF THE DRAWINGS

FIG. 1 shows a detection diagram of a plurality of bile acids simultaneously detected by liquid chromatography-tandem mass spectrometry in Example 1;

FIG. 2 shows a LASSO regression analysis diagram in Example 1;

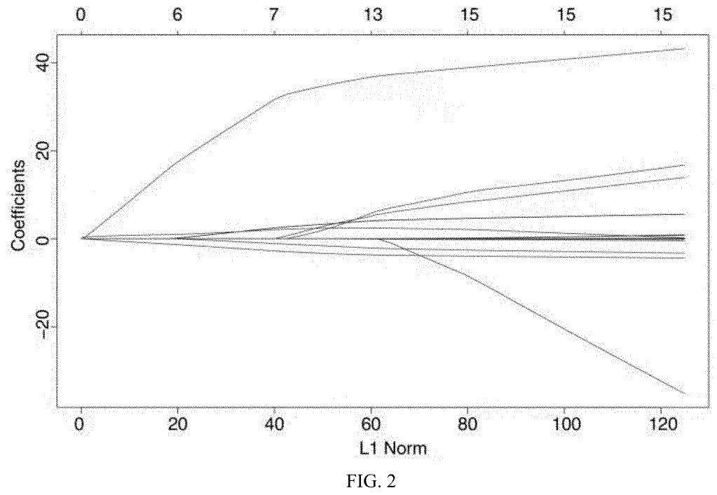

FIG. 3 shows a PLS-DA analysis diagram of an evaluation model 1 (BAS) for degree of liver fibrosis in Example 1;

FIG. 4 shows a PLS-DA analysis diagram of an evaluation model 2 (BASA) for degree of liver fibrosis in Example 1;

FIG. 5 shows a comparison diagram of the evaluation model 1 (BAS) for degree of liver fibrosis, the evaluation model 2 (BASA) for degree of liver fibrosis in Example 1, as well as ROC curves of the existing APRI and FIB.4;

FIG. 6 shows a ROC curve graph for the evaluation of clinical application of an evaluation model 1 (BAS) for degree of liver fibrosis in Example 2;

FIG. 7 shows a ROC curve graph for the evaluation of clinical application of an evaluation model 2 (BASA) for degree of liver fibrosis in Example 2.

### DETAILED DESCRIPTION OF THE EMBODIMENTS

The present invention will be further described in detail with reference to the accompanying drawings and examples hereafter. It should be indicated that the following examples are aimed at facilitating the understanding to the present invention, but not constructed as limiting the present invention. The reagents used in the examples are products known in the art and are commercially available.

### Example 1 Construction of an Evaluation Model for Degree of Liver Fibrosis

1. Data of Cases

152 cases (case group) of chronic hepatitis B patients receiving liver biopsy from Zhejiang Provincial People's Hospital from 2017 to 2019 were brought into this example. All the patients had no clinical manifestation of compensatory liver diseases and laboratory basis, nephrotic syndrome complicating pregnancy, and hematological system diseases.

2. Establishment of a Model

1) Liver biopsy and liver fibrosis staging of the cases:

All the selected patients received liver biopsy under abdomen ultrasound guidance. Liver biopsy was performed by percutaneous liver puncture for 1 s; a specimen was immediately put to a plastic specimen tube and frozen after being collected, then sent for examination. Liver tissue was put to a plastic embedding box and immobilized with neutral formalin, and dehydrated by gradient ethanol, subjected to transparency treatment by xylene, immersed and embedded by paraffin wax, then sliced, and stained by hematoxylin-eosin and subjected to reticular fiber staining. The quality evaluation of the liver tissue specimen and pathological diagnosis of the liver tissue were completed by an experienced pathologist independently. Liver histopathologic diagnosis is reference to the consensus on the evaluation of diagnosis and therapeutic effect of liver fibrosis in 2002; and the liver histopathologic fibrosis includes five stages, namely, S0, S1, S2, S3 and S4. The patients were divided into two groups according to the grading results, namely, the stage 1-2 (S0-S2) fibrosis and the stage 3-4 (S3-S4) fibrosis. There were 98 cases in the stage 1-2 fibrosis group, and 53 cases in the stage 3-4 fibrosis group.

2) Biochemical Detection of Blood, Liver Functions, 15 Bile Acids, and PLT:

4 ml whole blood was collected after fasting for 8 h, and centrifuged for 10 mins at 3500 r/min to collect blood serum; ALB, ALT, AST, GGT, ALP, TBIL, DBIL, IBIL, GLU, RBC, WBC and HGB were analyzed by a Roche cobas c702 fully automatic biochemical analyzer and the matching reagents; and APRI and FIB-4 were calculated; a plurality of bile acids were simultaneously analyzed by an AB SCIEX Triple Quad4500MD liquid chromatography-tandem mass spectrometer and the matching reagents; and LSM was collected; PLT was analyzed by a SYSME XE-2100 automatic blood cell analyzer and the matching reagents; three levels of quality control materials were detected every batch for indoor quality control; the (Out Of Control) rules are subjected to 13S, 22S and R4S.

3) Statistical Method:

R language software was used to process the data. Based on the fibrosis grouping of the patients, a multiple analysis method (PCA, ANOVA and PLS-DA) was used to judge the patient's degree of liver fibrosis, and LASSO regression analysis was performed on the all detection results to establish a same grading mathematical model for liver fibrosis, and inclusion and exclusion criteria of the independent variables were respectively P<0.05 and P>0.10; and the regression model efficiency was evaluated by a calibration curve and a ROC curve method.

3. Result Analysis:

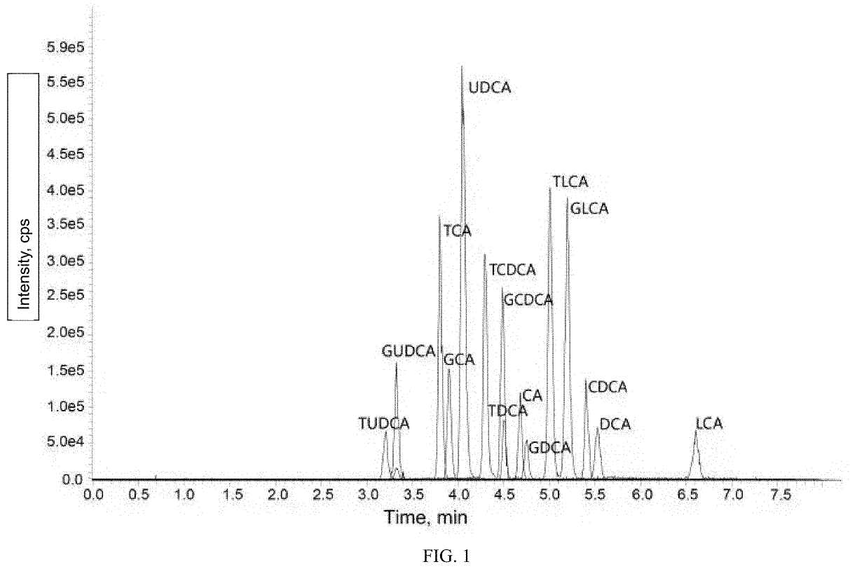

1) The detection method provided in CN2020101870483 was taken as a method for simultaneously detecting 15 bile acids by liquid chromatography-tandem mass spectrometry. The detection diagram is shown in FIG. 1

2) Based on LASSO, it is prompted that 15 bile acids have a distinct correlation to the occurrence of obvious liver fibrosis; and ALB, ALT, AST, GGT, ALP, TBIL, DBIL, IBIL, GLU, RBC, WBC, HGB and PLT also have a correlation to the occurrence of obvious liver fibrosis. The analysis result is shown in Table 1.

TABLE 1
Comparison of the detection results between the stage 1-2 (S0-S2)
liver fibrosis and the stage 3-4 (S3-S4) liver fibrosis

| | | | | 95% CI | |
|---|---|---|---|---|---|
| Indicator | β | OR | p-value | Lower | Upper |
| ALB | −0.110 | 0.896 | 0.001 | 1.166 | 4.626 |
| ALT | −0.017 | 0.983 | 0.566 | −12.998 | 7.142 |
| AST | 0.032 | 1.032 | 0.060 | −30.683 | 0.675 |
| GGT | 0.020 | 1.020 | 0.018 | −61.066 | −6.083 |
| ALP | 0.002 | 1.002 | 0.005 | −37.182 | −7.067 |
| TBIL | −0.141 | 0.868 | 0.003 | −8.823 | −1.852 |
| DBIL | 0.487 | 1.627 | 0.001 | −3.667 | −1.004 |
| IBIL | 0.137 | 1.147 | 0.010 | −5.760 | −0.807 |
| GLU | 0.577 | 1.781 | 0.001 | −0.943 | −0.239 |
| RBC | 0.137 | 1.146 | 0.196 | −0.130 | 0.619 |
| WBC | 0.190 | 1.209 | 0.641 | −0.542 | 0.875 |
| HGB | −0.013 | 0.987 | 0.076 | −0.940 | 18.552 |
| PLT | −0.010 | 0.990 | 0.000 | 27.841 | 68.440 |
| UDCA | 5.568 | 261.887 | 0.135 | −0.391 | 0.054 |
| GUDCA | −3.289 | 0.037 | 0.126 | −0.866 | 0.109 |
| TUDCA | 16.741 | 1.864 × 10⁷ | 0.025 | −0.168 | −0.012 |
| CA | −0.432 | 0.649 | 0.870 | −0.466 | 0.394 |
| GCA | 0.767 | 2.153 | 0.002 | −2.262 | −0.514 |
| TCA | 0.207 | 1.230 | 0.002 | −1.081 | −0.259 |
| CDCA | 0.103 | 1.108 | 0.333 | −0.723 | 0.247 |
| GCDCA | −0.155 | 0.856 | 0.014 | −6.194 | −0.723 |
| TCDCA | 0.894 | 2.445 | 0.006 | −2.508 | −0.452 |
| DCA | −4.394 | 0.012 | 0.047 | 0.003 | 0.398 |
| GDCA | −0.075 | 0.927 | 0.348 | −0.498 | 0.177 |
| TDCA | 0.003 | 1.003 | 0.434 | −1.859 | 0.806 |
| LCA | 43.177 | 5.643 × 10¹⁶ | 0.022 | −0.050 | −0.004 |
| GLCA | 13.907 | 1.0958 × 10⁶ | 0.192 | −0.024 | 0.005 |
| TLCA | −35.188 | 0.000 | 0.036 | −0.005 | 0.000 |

The higher the OR value is, the greater the impact of the people with stage 3-4 (S3-S4) on the indicator relative to the people with stage 1-2 (S0-S2), and the more obvious the indicator exposure is.

It can be seen from Table 1 that the bile acids, UDCA, TUDCA, LCA, GLCA, GCA and TCDCA have a particularly high correlation to the occurrence of obvious liver fibrosis; of which, LCA has the highest correlation with an OR value being up to 5.643×10¹⁶ and a very high weight; the second one is TUDCA with an OR value being up to 1.864×10⁷; the third one is GLCA with an OR value being up to 1.0958×10⁶; then followed by UDCA, TCDCA, GCA and TCA; weights of the above indicators are greater than other liver index values. By comparison, the correlation of the 15 bile acids to the occurrence of obvious liver fibrosis is apparently higher than other liver index values. As can be seen, it is of obvious advantage to establish an evaluation model for the degree of liver fibrosis by bile acids.

The degree of the concentration of bile acid to the degree of liver fibrosis can by represented by an OR value in Table 1. Certainly, the correlation may be distinguished by a β value, a p-value, or the like in Table 1; and OR value is the most visualized and distinct. For example, LCA has an OR value being up to 5.643×10¹⁶ which is 10¹⁶ folds of other bile acids; with such a high correlation, it can be understood that LCA concentration can be detected independently to judge the degree of liver fibrosis; for another example, TUDCA has an OR value being up to 1.864×10⁷ which is 10⁷ folds of other bile acids; GLCA has an OR value being up to 1.0958×10⁶, followed by UDCA, TCDCA, GCA, and TCA. The single detection of the concentration of a bile acid with a high correlation can be completely used to judge the degree of liver fibrosis, thus distinguishing the people with stage 1-2 liver fibrosis (S0-S2) and the people with stage 3-4 liver fibrosis (S3-S4).

Multi-factor regression analysis was performed on the correlation of the measured concentrations of the patient's 15 bile acids to the occurrence of obvious liver fibrosis to establish an evaluation model 1 (BAS) for the degree of liver fibrosis: BAS=43.18×LCA+16.74×TUDCA+13.91×GLCA+5.57×UDCA+0.89×TCDCA+0.77×GCA+0.21×TCA+0.1×CDCA+0×TDCA−0.08×GDCA−0.16×GCDCA−0.43×CA−3.29×GUDCA−4.39×DCA−35.19×TLCA.

Moreover, to further improve the detection sensitivity and specificity, DBIL, RBC, GLU, WBC, GGT, ALT, TBIL, AST and IBIL were picked according to the degree of correlation to the occurrence of obvious liver fibrosis and subjected to multi-factor regression analysis together with the measured concentrations of the patient's 15 bile acids, thus establishing an evaluation model 2 (BASA) for the degree of liver fibrosis: BASA=61.92×LCA+38.53×TUDCA+4.66×UDCA+2.14×TCA+0.9×GDCA+0.42×DBIL+0.27×RBC+0.26×GCDCA+0.24×CDCA+0.22×GLU+0.19×WBC+0.03×GGT+0.02×ALT+0.02×TBIL+0.01×AST−0.04×IBIL−0.05×TDCA−0.16×CA−0.67×GCA−0.8×GLCA−1.51×TCDCA−3.77×GUDCA−5.96×DCA−16.07×TLCA.

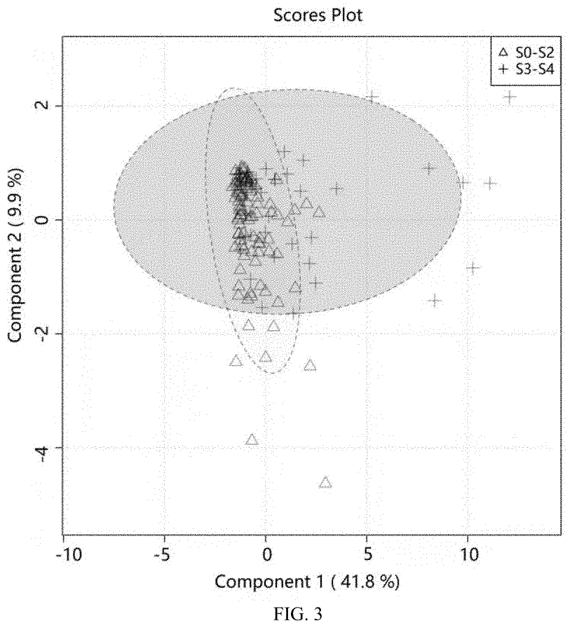

2) PLS-DA analysis diagram of the evaluation model 1 (BAS) for the degree of liver fibrosis is shown in FIG. 3. As can be seen, the people with stage 1-2 (S0-S2) liver fibrosis and the people with stage 3-4 (S3-S4) liver fibrosis may be completely separated by the model, namely, the concentrations of the 15 bile acids.

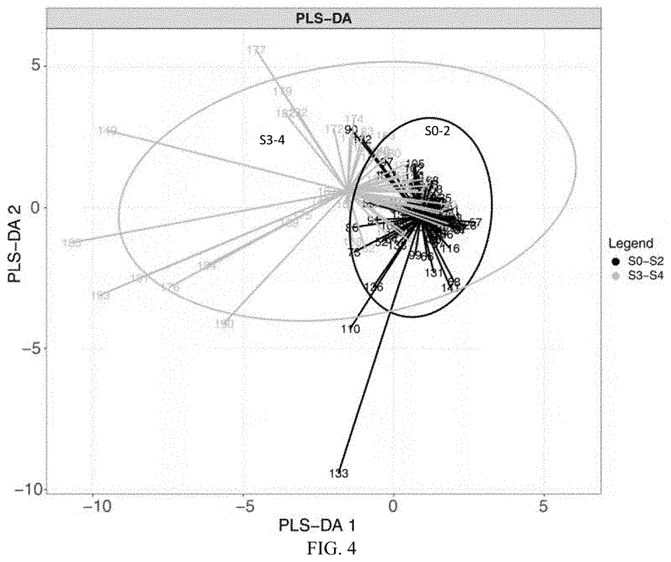

2) PLS-DA analysis diagram of the evaluation model 2 (BASA) for the degree of liver fibrosis is shown in FIG. 4. As can be seen, the people with stage 1-2 (S0-S2) liver fibrosis and the people with stage 3-4 (S3-S4) liver fibrosis may be completely separated by the model; and the separation effect is better than that of the evaluation model 1 for the degree of liver fibrosis.

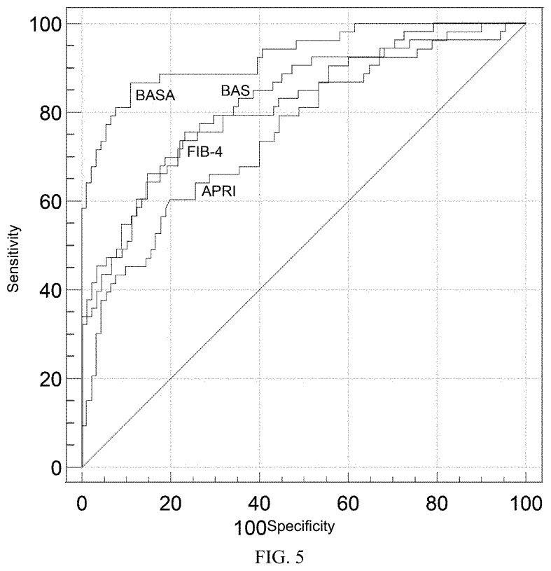

3) ROC curves of the new and previous models were compared by means of the evaluation model 1 (BAS) for degree of liver fibrosis, the evaluation model 2 (BASA) for degree of liver fibrosis newly established herein, as well as APRI and FIB.4. The results are shown in FIG. 5, Tables 2 and 3.

TABLE 2
Comparison results of the ROC curves among BAS, BASA, FIB.4 and APRI

| Indicator | BAS | BASA | FIB-4 | APRI |
|---|---|---|---|---|
| Areas Under The | 0.834 | 0.928 | 0.802 | 0.757 |
| ROC Curve (AUC) | | | | |
| Standard error a | 0.0349 | 0.0231 | 0.0403 | 0.0419 |
| 95% confidence | 0.763-0.891 | 0.873-0.964 | 0.727-0.865 | 0.679-0.825 |
| interval b | | | | |
| Z statistics | 9.578 | 18.506 | 7.498 | 6.146 |
| Significance level | <0.0001 | <0.0001 | <0.0001 | <0.0001 |
| P (area = 0.5) | | | | |
| Youden index | 0.5239 | 0.7580 | 0.5127 | 0.4038 |
| Relevant standard | <0.63 | <0.63 | >1.72 | >0.70 |
| Sensibility | 75.47 | 86.79 | 66.04 | 60.38 |
| Specificity | 76.92 | 89.01 | 85.23 | 80.00 |

TABLE 3
Comparison result of sensitivity and specificity
among BAS, BASA, FIB-4 and APRI

| Indicator | Standard | Sensibility | 95% CI | Specificity | 95% CI | +LR | −LR |
|---|---|---|---|---|---|---|---|
| BAS | ≤0.63 | 75.47 | 61.7-86.2 | 76.92 | 66.9-85.1 | 3.27 | 0.32 |
| BASA | ≤0.63 | 86.79 | 74.7-94.5 | 89.01 | 80.7-94.6 | 7.90 | 0.15 |
| FIB-4 | >1.72 | 66.04 | 51.7-78.5 | 85.23 | 76.1-91.9 | 4.47 | 0.40 |
| APRI | >0.70 | 60.38 | 46.0-73.5 | 80.00 | 70.2-87.7 | 3.02 | 0.50 |

It can be seen from Tables 2-3 and FIG. 5 that the evaluation model 1 (BAS) for the degree of liver fibrosis has an AUC value of 0.834, a sensitivity of 75.47% and a specificity of 76.92; the evaluation model 2 (BASA) for the degree of liver fibrosis has an AUC value of 0.928, a sensitivity of 86.79% and a specificity of 89.01%; the above results are much higher than the detection efficiency of the existing model APRI (AUC value: 0.757), and FIB.4 (AUC value: 0.802).

It can be further seen from Tables 2-3 that according to the calculation results of the BAS and BASA of the people with stage 1-2 (S0-S2) liver fibrosis and the people with stage 3-4 (S3-S4) liver fibrosis, the standard of BAS and BASA is ≤0.63; when BAS is greater than 0.63, the liver fibrosis is a stage 3-4 liver fibrosis; when BAS is lower than 0.63, the liver fibrosis is a stage 1-2 liver fibrosis; when BASA is greater than 0.63, the liver fibrosis is a stage 3-4 liver fibrosis; and when BASA is lower than 0.63, the liver fibrosis is a stage 1-2 liver fibrosis.

### Example 2 Clinical Application of BAS and BASA of the Evaluation Model for the Degree of Liver Fibrosis

32 cases of chronic hepatitis B patients from Zhejiang Provincial People's Hospital in October 2021 were selected in this example, and divided into two groups, 16 cases in each group. The degree of liver fibrosis was respectively evaluated by the evaluation model 1 (BAS) for the degree of liver fibrosis and the evaluation model 2 (BASA) for the degree of liver fibrosis provided in Example 1. The evaluation results were compared with the final results of the liver biopsy, and AUC values of the BAS and BASA were verified and calculated. The evaluation results of the degree of liver fibrosis are shown in Tables 1-2.

TABLE 1
Comparison of BAS evaluation results

| Specimen | Liver biopsy result | BAS evaluation result |
|---|---|---|
| Specimen 1 | S0-S2 | S0-S2 |
| Specimen 2 | S0-S2 | S0-S2 |
| Specimen 3 | S0-S2 | S0-S2 |
| Specimen 4 | S0-S2 | S0-S2 |
| Specimen 5 | S0-S2 | S0-S2 |
| Specimen 6 | S0-S2 | S0-S2 |
| Specimen 7 | S0-S2 | S0-S2 |
| Specimen 8 | S0-S2 | S0-S2 |
| Specimen 9 | S0-S2 | S0-S2 |
| Specimen 10 | S0-S2 | S0-S2 |
| Specimen 11 | S0-S2 | S0-S2 |
| Specimen 12 | S0-S2 | S0-S2 |
| Specimen 13 | S0-S2 | S0-S2 |
| Specimen 14 | S3-S4 | S3-S4 |
| Specimen 15 | S0-S2 | S3-S4 |
| Specimen 16 | S0-S2 | S3-S4 |

TABLE 2
Comparison of BASA evaluation results

| Specimen | Liver biopsy result | BAS evaluation result |
|---|---|---|
| Specimen 1 | S0-S2 | S0-S2 |
| Specimen 2 | S0-S2 | S0-S2 |
| Specimen 3 | S0-S2 | S0-S2 |
| Specimen 4 | S0-S2 | S0-S2 |
| Specimen 5 | S0-S2 | S0-S2 |
| Specimen 6 | S0-S2 | S0-S2 |
| Specimen 7 | S0-S2 | S0-S2 |
| Specimen 8 | S0-S2 | S0-S2 |
| Specimen 9 | S0-S2 | S0-S2 |
| Specimen 10 | S0-S2 | S0-S2 |
| Specimen 11 | S0-S2 | S0-S2 |
| Specimen 12 | S0-S2 | S0-S2 |
| Specimen 13 | S0-S2 | S0-S2 |
| Specimen 14 | S0-S2 | S0-S2 |
| Specimen 15 | S0-S2 | S0-S2 |
| Specimen 16 | S3-S4 | S3-S4 |

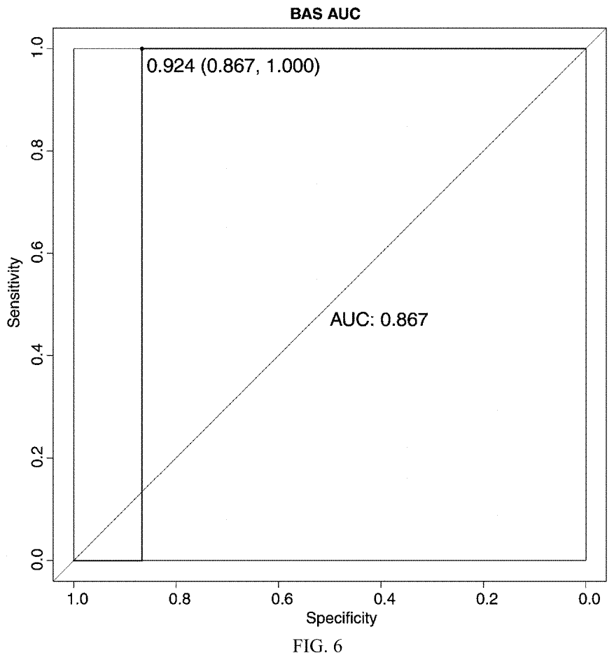

It can be seen from Table 1 that when BAS is used for evaluation, the evaluation results of the degree of fibrosis of the 15th and the 16th specimens in the 16 specimens are slightly higher; the evaluation results of other specimens are consistent with the results of the liver biopsy; the accuracy rate is 87.5% and the verification result of the AUC value is up to 0.924 (FIG. 6).

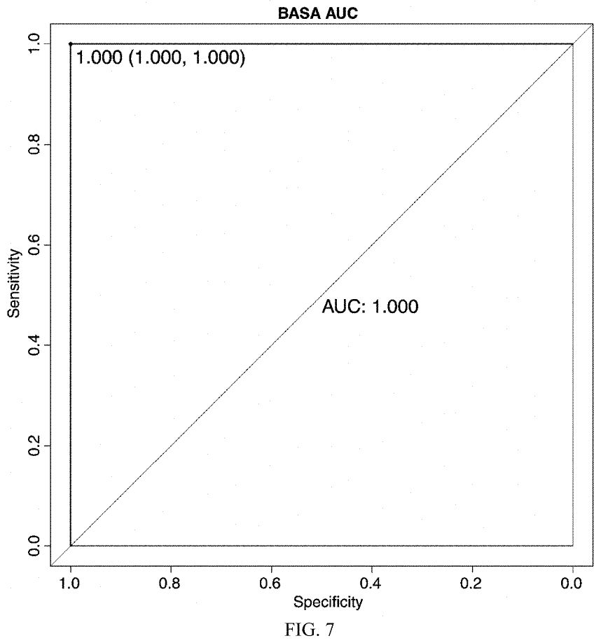

It can be seen from Table 2 that when BASA is used for evaluation, the evaluation results of the degree of fibrosis of the 16 specimens are completely consistent with the results of the liver biopsy; the accuracy rate is 100% and the verification result of the AUC value is up to 1.000 (FIG. 7).

The present invention is disclosed above, but the present invention is not limited thereto. Any person skilled in the art can make various alterations and modifications within the spirit and scope of the present invention. Therefore, the protection scope of the present invention should be subjected to the scope defined in the claims.

1. A method for evaluating degree of liver fibrosis, wherein, a degree or a level of liver fibrosis is evaluated by detecting a concentration of a bile acid in a sample.

2. The method of claim 1, wherein, the bile acid is selected from any one or more of GCA, TCA, CDCA, GCDCA, TCDCA, DCA, GDCA, TDCA, LCA, GLCA, TLCA, UDCA, GUDCA, TUDCA and CA.

3. The method of claim 1, wherein, the bile acid is selected from any one or more of UDCA, TUDCA, LCA, GLCA, GCA and TCDCA.

4. The method of claim 3, wherein, the bile acid is selected from any one or more of LCA, TUDCA and GLCA.

5. The method of claim 4, wherein, the bile acid is selected from any one or more of LCA and TUDCA.

6. The method of claim 5, wherein, the bile acid is selected from LCA.

7. The method of claim 6, wherein, a regression equation is constructed to calculate a predicted value of liver fibrosis, thus evaluating the degree of liver fibrosis, and the regression equation is as follows: BAS=43.18×LCA+16.74×TUDCA+13.91×GLCA+5.57×UDCA+0.89×TCDCA+0.77×GCA+0.21×TCA+0.1×CDCA+0×TDCA−0.08×GDCA−0.16×GCDCA−0.43×CA−3.29×GUDCA−4.39×DCA−35.19×TLCA, in which, BAS represents a predicted value of liver fibrosis, and wherein, the predicted value is correlated to the degree of liver fibrosis.

8. The method of claim 7, wherein, a regression equation is constructed to calculate a predicted value of liver fibrosis, thus evaluating the degree of liver fibrosis, and the regression equation is as follows: BASA=61.92×LCA+38.53×TUDCA+4.66×UDCA+2.14×TCA+0.9×GDCA+0.42×DBIL+0.27×RBC+0.26×GCDCA+0.24×CDCA+0.22×GLU+0.19×WBC+0.03×GGT+0.02×ALT+0.02×TBIL+0.01×AST−0.04×IBIL−0.05×TDCA−0.16×CA−0.67×GCA−0.8×GLCA−1.51×TCDCA−3.77×GUDCA−5.96×DCA−16.07×TLCA, in which, BASA represents a predicted value of liver fibrosis.

9. The method of claim 8, wherein, the regression equation has a value of BAS or BASA; when BAS is greater than 0.63, the liver fibrosis is a stage 3-4 liver fibrosis; when BAS is lower than 0.63, the liver fibrosis is a stage 1-2 liver fibrosis; when BASA is greater than 0.63, the liver fibrosis is a stage 3-4 liver fibrosis; and when BASA is lower than 0.63, the liver fibrosis is a stage 1-2 liver fibrosis.

10. A system for evaluating degree of liver fibrosis, comprising a data input/output interface and a data analysis unit, wherein the data input/output interface needs to input a concentration of at least one bile acid, after the concentration is analyzed by the data analysis unit, the data input/output interface outputs an evaluation result of the degree of liver fibrosis.

11. The system of claim 10, wherein, the bile acid comprises any one or more of GCA, TCA, CDCA, GCDCA, TCDCA, DCA, GDCA, TDCA, LCA, GLCA, TLCA, UDCA, GUDCA, TUDCA and CA.

12. The system of claim 11, wherein, the bile acid comprises any one or more of UDCA, TUDCA, LCA, GLCA, GCA and TCDCA.

13. The system of claim 12, wherein, the bile acid comprises any one or more of LCA, TUDCA and GLCA.

14. The system of claim 13, wherein, the bile acid comprises any one or more of LCA and TUDCA.

15. The system of claim 14, wherein, the bile acid comprises LCA.

16. The system of claim 15, wherein, the data analysis unit calculates a predicted value of liver fibrosis based on the regression equation, thus evaluating a degree of liver fibrosis.

17. The system of claim 16, wherein, the regression equation is as follows: BAS=43.18×LCA+16.74×TUDCA+13.91×GLCA+5.57×UDCA+0.89×TCDCA+0.77×GCA+0.21×TCA+0.1×CDCA+0×TDCA−0.08×GDCA−0.16×GCDCA−0.43×CA−3.29×GUDCA−4.39×DCA−35.19×TLCA, in which, BAS represents a predicted value of liver fibrosis.

18. The system of claim 17, wherein, the data input/output interface further needs to input a concentration/concentrations of one or more of DBIL, RBC, GLU, WBC, GGT, ALT, TBIL, AST and IBIL.

19. The system of claim 18, wherein, the regression equation is as follows: BASA=61.92×LCA+38.53×TUDCA+4.66×UDCA+2.14×TCA+0.9×GDCA+0.42×DBIL+0.27×RBC+0.26×GCDCA+0.24×CDCA+0.22×GLU+0.19×WBC+0.03×GGT+0.02×ALT+0.02×TBIL+0.01×AST−0.04×IBIL−0.05×TDCA−0.16×CA−0.67×GCA−0.8×GLCA−1.51×TCDCA−3.77×GUDCA−5.96×DCA−16.07×TLCA, in which, BASA represents a predicted value of liver fibrosis.

20. The system of claim 19, wherein, the regression equation has a value of BAS or BASA; when BAS is greater than 0.63, the liver fibrosis is a stage 3-4 liver fibrosis; when BAS is lower than 0.63, the liver fibrosis is a stage 1-2 liver fibrosis; when BASA is greater than 0.63, the liver fibrosis is a stage 3-4 liver fibrosis; and when BASA is lower than 0.63, the liver fibrosis is a stage 1-2 liver fibrosis.
